(19) United States
(12) Patent Application Publication
(10) Pub. No.: US 2010/0331590 A1
(43) Pub. Date: Dec. 30, 2010

(54) PRODUCTION OF LIGHT OLEFINS AND AROMATICS

(76) Inventors: Debarshi Majumder, Chicago, IL (US); Bryan K. Glover, Algonquin, IL (US)

Correspondence Address: HONEYWELL/UOP;PATENT SERVICES, 101 COLUMBIA DRIVE, P O BOX 2245 MAIL STOP AB/2B, MORRISTOWN, NJ, 07962, US

(21) Appl. No.: 12/491,344

(22) Filed: Jun. 25, 2009

(51) Int. Cl. C07C 4/06

(52) U.S. Cl. 585/315; 585/319; 585/324

(57) ABSTRACT

Processes for the conversion of both straight- or branched-chain (e.g., paraffinic) as well as cyclic (e.g., naphthenic) hydrocarbons of a hydrocarbon feedstock into value added product streams are disclosed. The processes involve the use of both dehydrogenation and olefin cracking to produce both light olefins and aromatics in varying proportions depending on the feedstock composition and particular processing scheme. The processes are especially applicable to naphtha feedstocks comprising paraffins and naphthenes in the C₅-C₁₁ carbon number range.

### FIELD OF THE INVENTION

The present invention relates to processes for producing light olefins, particularly at high propylene:ethylene molar ratios, by paraffin dehydrogenation and olefin cracking of hydrocarbon feed streams such as naphtha. Aromatics are recovered in combination with the light olefins.

### DESCRIPTION OF RELATED ART

Ethylene and propylene are important products for the production of polyethylene and polypropylene, which are two of the most common plastics manufactured today. Additional uses for ethylene and propylene include the production of commercially important monomers, namely vinyl chloride, ethylene oxide, ethylbenzene, and alcohols. Ethylene and propylene have traditionally been produced through steam cracking or pyrolysis of hydrocarbon feedstocks such as natural gas, petroleum liquids, and carbonaceous materials (e.g., coal, recycled plastics, and organic materials).

An ethylene plant involves a very complex combination of reaction and gas recovery systems. Feedstock is charged to a thermal cracking zone in the presence of steam at effective conditions to produce a pyrolysis reactor effluent gas mixture. The mixture is then stabilized and separated into purified components through a sequence of cryogenic and conventional fractionation steps. Ethylene and propylene yields from steam cracking and other processes may be improved using known methods for the metathesis or disproportionation of C₄ and heavier olefins, in combination with a cracking step in the presence of a zeolitic catalyst, as described, for example, in U.S. Pat. No. 5,026,935 and U.S. Pat. No. 5,026,936. The cracking of olefins in hydrocarbon feedstocks comprising C₄ mixtures from refineries and steam cracking units is described in U.S. Pat. No. 6,858,133; U.S. Pat. No. 7,087,155; and U.S. Pat. No. 7,375,257.

Paraffin dehydrogenation represents an alternative route to light olefins and is described in U.S. Pat. No. 3,978,150 and elsewhere. More recently, the desire for alternative, non-petroleum based feeds for light olefin production has led to the use of oxygenates such as alcohols and, more particularly, methanol, ethanol, and higher alcohols or their derivatives. Methanol, in particular, is useful in a methanol-to-olefin (MTO) conversion process described, for example, in U.S. Pat. No. 5,914,433. The yield of light olefins from such a process may be improved using olefin cracking to convert some or all of the C₄⁺ product of MTO in an olefin cracking reactor, as described in U.S. Pat. No. 7,268,265. Other processes for the generation of light olefins involve high severity catalytic cracking of naphtha and other hydrocarbon fractions. A catalytic naphtha cracking process of commercial importance is described in U.S. Pat. No. 6,867,341.

Despite the variety of methods for generating light olefins industrially, the demand for ethylene and propylene is outpacing the capacity of these conventional processes. Moreover, further demand growth for light olefins is expected. A need therefore exists for new methods that can economically increase light olefin yields from existing sources of both straight-run and processed hydrocarbon streams.

### SUMMARY OF THE INVENTION

The present invention is associated with the discovery of processes that provide not only light olefins in high yields, but also aromatic hydrocarbons (e.g., C₆-C₈ aromatics, namely benzene, toluene, and xylenes) that are themselves valuable, for example, as precursors of polymers (e.g., polystyrene, polyesters, and others) for a wide range of applications. Importantly, the inventive processes have the capability of generating a light olefin product having a high propylene:ethylene molar ratio compared to conventional technologies such as catalytic naphtha cracking. This is especially desirable in view of current trends indicating an increase in the demand for propylene relative to that of ethylene. Processes described herein have the further advantage of flexibility in tailoring feedstocks of varying characteristics to a product slate with desired proportions of light olefins and aromatics, thereby optimizing the overall product value for a given feed composition and individual product prices.

Embodiments of the invention are directed to processes for the conversion of both straight- or branched-chain (e.g., paraffinic) as well as cyclic (e.g., naphthenic) hydrocarbons of a hydrocarbon feedstock into value added product streams. The processes involve the use of both dehydrogenation and olefin cracking zones, either in separate reactors or within a single vessel, to produce both light olefins and aromatics in varying proportions depending on the feedstock composition and particular processing scheme. The processes are especially applicable to naphtha feedstocks comprising paraffins and naphthenes in the C₅-C₁₁ carbon number range. In a preferred embodiment, the catalyst used in the dehydrogenation zone comprises zirconia to effectively convert such hydrocarbons to corresponding olefins and aromatics.

Advantageously, a wide range of naphtha qualities are efficiently converted, through dehydrogenation and subsequent olefin cracking, to high value end products in proportions governed at least partly by the hydrocarbon feedstock composition. For example, a rich naphtha feedstock having a relatively high content of naphthenes can provide a significant aromatic product yield, in addition to the light olefins propylene and ethylene. This processing flexibility, which allows the product slate to be tailored to a particular hydrocarbon feedstock such as naphtha, is associated with the optional use of upstream catalytic reforming, as described, for example, in U.S. Pat. No. 4,119,526; U.S. Pat. No. 4,409,095; and U.S. Pat. No. 4,440,626. A preferred type of catalytic reforming involves continuous catalyst regeneration (CCR) in conjunction with a moving catalyst bed system, as described, for example, in U.S. Pat. No. 3,647,680; U.S. Pat. No. 3,652,231, U.S. Pat. No. 3,692,496; and U.S. Pat. No. 4,832,921. Embodiments of the invention are therefore directed to processes that combine catalytic reforming with dehydrogenation and olefin cracking, as well as processes that utilize only the latter two conversion zones if warranted for a particular feedstock. In either case, the use of a zirconia catalyst for dehydrogenation is preferred.

The inventive processes allow for the production of both olefins and aromatics in high yields from a wide variety of hydrocarbon feedstocks, and particularly those comprising naphtha boiling range hydrocarbons, either as straight-run or processed fractions. Product yields in the olefin cracking effluent and separated products (e.g., the light olefin product and aromatic product) are often favorable to alternative technologies in terms of the propylene:ethylene molar ratio and other properties.

These and other embodiments, and their associated advantages, relating to the present invention are apparent from the following Detailed Description.

### BRIEF DESCRIPTION OF THE DRAWING

FIG. 1 depicts a representative process involving the use of catalytic dehydrogenation and olefin cracking zones, optionally with upstream catalytic reforming, for the production of light olefins and aromatics.

FIG. 1 is to be understood to present an illustration of the invention and/or principles involved. Details including pumps, compressors, heaters and heat exchangers, reboilers, condensers, instrumentation and control loops, and other items not essential to the understanding of the invention are not shown. As is readily apparent to one of skill in the art having knowledge of the present disclosure, methods for producing light olefins and aromatics according to various other embodiments of the invention have configurations, equipment, and operating parameters determined, in part, by the specific hydrocarbon feedstocks, products, and product quality specifications.

### DETAILED DESCRIPTION

Embodiments of the invention relate to the use of dehydrogenation in combination with olefin cracking of a hydrocarbon feedstock to provide high yields of both light olefins, particularly propylene and ethylene, and aromatics, particularly benzene, toluene, and xylenes. A representative feedstock comprises naphtha (e.g., straight-run naphtha), comprising hydrocarbons boiling in the range from about 100° C. (212° F.) to about 180° C. (356° F.). Other feedstocks comprising hydrocarbons boiling in this range, including processed hydrocarbon fractions (e.g., obtained from hydrocracking or fluid catalytic cracking) or synthetic naphtha, are also suitable. Hydrocarbon feedstocks of interest therefore generally have an initial boiling point, or distillation “front-end,” temperature generally in the range from about 75° C. (167° F.) to about 120° C. (248° F.), and often from about 85° C. (185° F.) to about 110° C. (230° F.), and a distillation end point temperature generally from about 138° C. (280° F.) to about 216° C. (420° F.), and often from about 160° C. (320° F.) to about 193° C. (380° F.), according to method ASTM D-86.

Preferred feedstocks such as those comprising naphtha contain both cyclic and non-cyclic hydrocarbons in the C₅-C₁₁ carbon number range, and often contain hydrocarbons having each of these carbon numbers; for example, a representative naphtha contains at least some quantity of paraffins having 5, 6, 7, . . . , 11 carbon atoms (e.g., pentane, hexane, heptane, . . . , undecane), in addition to cycloalkanes having 5, 6, 7, . . . , 11 carbon atoms (e.g., cyclopentane, cyclohexane, 1,2-dimethylcyclopentane, . . . , 1,2-diethyl, 3-methyl-cyclohexane), as well as aromatics having 6, 7, 8, . . . , 11 carbon atoms (e.g., benzene, toluene, xylenes, . . . , 1,2-diethyl, 3-methyl-cyclohexane). Naphtha, including a straight-run naphtha fraction, that is suitable as a hydrocarbon feedstock, or a component of the feedstock, generally comprises a total amount of paraffins, both straight- and branched-chain, in the C₅-C₁₁ carbon number range from about 40% to about 80% by weight. The naphthenes and aromatics in this carbon number range are generally present in naphtha in total amounts from about 20% to about 50% by weight and from about 5% to about 30% by weight, respectively. Naphtha may also contain a total amount of olefins in the C₅-C₁₁ carbon number range from about 5% to about 25%, particularly in the case of naphtha fractions derived from processes carried out in a hydrogen deficient environment, such as fluid catalytic cracking, thermal cracking, or steam cracking.

A particular naphtha may be characterized as “rich” or “lean” depending on the amount of naphthenes and aromatics present, relative to the amount of paraffins. The composition of a particular naphtha is an important consideration in determining whether a hydrocarbon feedstock containing such naphtha should be subjected to catalytic reforming upstream of dehydrogenation and olefin cracking, according to representative embodiments of the invention. Particularly relevant is the quantity of cyclopentanes and alkylcyclopentanes, which are advantageously converted via catalytic reforming to valuable aromatics. Reforming is desirable, for example, in case of a rich naphtha comprising a relatively high amount of cyclic hydrocarbons, including a total amount of cyclopentanes and alkylcyclopentanes from about 10% to about 25% by weight.

A naphtha reforming effluent as a hydrocarbon feedstock or feedstock component generally contains some unconverted paraffins in C₅-C₁₁ carbon number range. Relative to naphtha that has not undergone reforming, a naphtha reforming effluent generally contains significantly greater amounts of aromatics, for example in the range from about 30% to about 70% by weight. Also, as a result of reforming reactions and particularly paraffin cyclization, the distillation endpoint of a naphtha reforming effluent is normally significantly increased, for example, to a representative temperature from about 152° C. (305° F.) to about 241° C. (465° F.).

Embodiments of the invention are therefore directed to processes comprising dehydrogenating a hydrocarbon feedstock and subjecting the dehydrogenation effluent to olefin cracking. The hydrocarbon feedstock preferably comprises, or in some cases consists essentially of (i.e., without additional feedstock components that alter its basic properties), naphtha. Alternatively, a hydrocarbon feedstock may comprise, or consist essentially of, a naphtha reforming effluent as discussed above, or possibly a mixture of naphtha and a naphtha reforming effluent (e.g., a lean naphtha and a naphtha reforming effluent obtained from reforming a rich naphtha). Feedstocks may comprise or consist essentially of other components including higher boiling distillate fractions such as atmospheric and vacuum gas oils. The feedstocks may be combined with other components, including those generated in processes of the invention and recycled upstream of the dehydrogenation zone.

Hydrocarbon feedstocks such as those comprising naphtha and/or a naphtha reforming effluent as discussed above are dehydrogenated such that paraffins in the feedstock, and particularly those in the C₅-C₁₁ carbon number range, are converted to olefins in the dehydrogenation zone and exit this zone or reactor in the dehydrogenation effluent. Suitable dehydrogenation conditions in the dehydrogenation zone or reactor include an average dehydrogenation catalyst bed temperature from about 450° C. (842° F.) to about 700° C. (1292° F.), and an absolute pressure from about 50 kPa (7 psia) to about 2 MPa (290 psia), preferably from about 100 kPa (15 psia) to about 1 MPa (145 psia).

Preferably, the dehydrogenation catalyst present in the dehydrogenation zone or reactor comprises zirconia, which is effective for the dehydrogenation of the intermediate boiling range (e.g., C₅-C₁₁) paraffins in the hydrocarbon feedstock, and particularly in the naphtha and/or naphtha reforming effluent component(s) thereof, as discussed above, to corresponding olefins. Without being bound by theory, zirconia-based catalysts provide a low cost means of readily dehydrogenating paraffins in this carbon number range at near equilibrium conversion levels to corresponding carbon number olefins. A representative dehydrogenation catalyst generally contains zirconia in an amount of at least about 40% (e.g., from about 50% to about 90%) by weight. Other possible components of the dehydrogenation catalyst include other metal oxides that can stabilize the zirconia, including oxides of one or more metals selected from the group consisting of scandium, yttrium, lanthanum, cerium, actinium, calcium, and magnesium. If used, the metal oxide(s) other than zirconia is/are generally present in an amount of at most about 10% by weight of the dehydrogenation catalyst. Also, suitable binders and fillers such as alumina, silica, clays, aluminum phosphate, etc. may be incorporated into the catalyst in a total amount of generally at most about 50% by weight of the dehydrogenation catalyst. The dehydrogenation catalyst is typically present in a dehydrogenation reactor as a fluidized bed, with a short (e.g., from about 10 minutes to about 100 minutes) time in which the catalyst is used in dehydrogenation processing prior to regeneration. Alternatively, a moving bed, fixed bed, or other type of catalyst bed may be employed.

The dehydrogenation effluent exiting the dehydrogenation reactor or zone comprises olefins as a result of dehydrogenation. At least a portion of these olefins (e.g., in the C₅ to C₁₁ carbon number range) are then cracked in an olefin cracking zone or reactor to provide an olefin cracking effluent comprising ethylene, propylene, and aromatics. The portion of olefins that are cracked may correspond to the conversion in the olefin cracking zone, for example, of C₅-C₁₁ olefins to propylene or ethylene. In alternative embodiments, not all of the dehydrogenation effluent, including the olefins contained therein, is passed to the olefin cracking zone or reactor. In this case, the portion of olefins that are cracked corresponds to the olefin cracking conversion multiplied by the fraction of olefins present in the dehydrogenation effluent that are actually passed to the olefin cracking reactor. According to some embodiments, as discussed below, the dehydrogenation effluent may be combined, prior to subsequent cracking of all or a portion of the olefins in the dehydrogenation effluent, with other products of the process, such as a selective hydrogenation reactor effluent and/or a recycled portion of a heavy hydrocarbon byproduct.

If the total dehydrogenation effluent is passed to the olefin cracking zone, it may be possible to locate both the dehydrogenation and olefin cracking zones (e.g., containing different beds of catalyst) within the same reactor. In many cases, however, separate reactors are desirable due to the different conditions, including reaction pressure, used in each of these zones. Representative conditions in the olefin cracking zone include an olefin cracking catalyst bed inlet temperature from about 400° C. (752° F.) to about 600° C. (1112° F.) and an absolute olefin partial pressure from about 10 kPa (1.5 psia) to about 200 kPa (29 psia). Olefin cracking is normally carried out in the presence of a fixed bed of catalyst at a liquid hourly space velocity (LHSV) from about 5 to about 30 hr⁻¹. The LHSV, closely related to the inverse of the reactor residence time, is the volumetric liquid flow rate over the catalyst bed divided by the bed volume and represents the equivalent number of catalyst bed volumes of liquid processed per hour. As described in U.S. Pat. No. 7,317,133, suitable catalysts for olefin cracking comprise crystalline silicates, and particularly those having the MEL or MFI structure type, which are bound with an inorganic binder. MFI crystalline silicates may be dealuminated as described in this reference.

Advantageously, the olefin cracking effluent comprises valuable light olefins in combination with aromatics. Propylene and ethylene are present in this effluent typically in an amount representing at least about 40% by weight of the feedstock (e.g., naphtha, naphtha reforming effluent, or combination thereof), and often in an amount representing from about 45% to about 65% by weight of the feedstock. The total amount of C₁-C₃ hydrocarbons typically represent from about 50% to about 75% by weight of the feedstock, meaning that a high proportion (e.g., at least about 85%, often from about 85% to about 92%) of the C₁-C₃ hydrocarbons are the highest-value propylene and ethylene hydrocarbons. Moreover, as discussed above, the propylene:ethylene molar ratio of the light olefins produced is generally favorable, especially in cases in which the value of propylene (e.g., in dollars per metric ton) exceeds that of ethylene. Normally, the propylene:ethylene molar ratio in the olefin cracking effluent is at least about 1.5:1 (e.g., in the range from about 1.5:1 to about 4:1), typically at least about 2:1 (e.g., in the range from about 2:1 to about 3.5:1), and often at least about 2.3:1 (e.g., in the range from about 2.3:1 to about 2.8:1).

The aromatics content of the olefin cracking effluent also enhances the value of this product, and particularly in embodiments, as discussed above, in which a naphtha reforming effluent is used as the hydrocarbon feedstock or a component thereof. Upstream reforming is especially beneficial in converting saturated cyclic hydrocarbons, particularly cyclopentanes and alkylcyclopentanes, to C₆⁺ aromatics, as these compounds are normally difficult to convert in a similar manner in the dehydrogenation zone. The use of a naphtha reforming effluent as a hydrocarbon feedstock therefore normally provides an olefin cracking effluent having valuable C₆-C₈ aromatics (benzene, toluene, and xylenes) present in an amount representing from about 20% to about 40% by weight of the feedstock. A straight run naphtha or other naphtha that is not subjected to reforming, as a hydrocarbon feedstock, typically provides an olefin cracking effluent having C₆-C₈ aromatics present in an amount from about 10% to about 25% by weight of the feedstock. In general, the yield of these aromatics, whether or not the feedstock is partially or completely subjected to upstream reforming, is in the range from about 10% to 50%, and often from about 10% to about 30%, by weight of the feedstock.

Recovery of the light olefins and aromatics in the olefin cracking reactor effluent into more purified products, such as a light olefin product and an aromatic product, may be accomplished using a number of separations, including distillation or fractionation, flash separation, solvent absorption/stripping, membrane separation, and/or solid adsorptive separation. Combinations of such separations are usually employed. According to particular embodiments, the olefin cracking effluent is fractionated into low boiling and high boiling (e.g., overhead and bottoms) fractions of a distillation column, with these fractions being enriched, respectively, in light olefins (propylene and ethylene) and aromatics. The light olefin product may be taken as the low boiling fraction without further purification, or otherwise additional separations can be performed to provide one or more light olefin product(s) containing propylene and/or ethylene at a high purity.

The aromatic product may be taken as the high boiling fraction from the fractionation column, but often it is desirable to separate an aromatic product from this high boiling fraction that is further enriched in aromatic hydrocarbon content. Various methods for recovering aromatics from impure hydrocarbon streams are known, with representative conventional methods utilizing selective absorption of aromatics into physical solvents such as propylene carbonate, tributyl phosphate, methanol, or tetrahydrothiophene dioxide (or tetramethylene sulfone). Other physical solvents include alkyl- and alkanol-substituted heterocyclic hydrocarbons such as alkanolpyridines (e.g., 3-(pyridin-4-yl)-propan-1-ol) and alkylpyrrolidones (e.g., n-methylpyrrolidone), as well as dialkylethers of polyethylene glycol.

The separation of an aromatic product from the high boiling fraction generates a heavy hydrocarbon byproduct, typically containing paraffins, olefins, and possibly alkylcyclopentanes (especially in the absence of a reforming step). Some or all of the heavy hydrocarbon byproduct may be recycled to the olefin cracking zone, for example by combining it with the dehydrogenation effluent, to improve the overall conversion of the process and yields of desired products. In many cases, a non-recycled portion of the heavy hydrocarbon byproduct is purged in order to prevent an excessive accumulation of one or more unwanted, heavy hydrocarbon compounds. In alternative embodiments, all or a portion of the heavy hydrocarbon byproduct that is not sent to the olefin cracking zone is instead returned to dehydrogenation zone together with the hydrocarbon feedstock entering this zone.

The product fractionator, normally a depropanizer that separates C₃ and lighter hydrocarbons in the overhead, may also generate, in addition to the low and high boiling fractions, an intermediate boiling fraction comprising olefins in the C₅-C₁₁, carbon number range (e.g., containing hydrocarbons having each of these carbon numbers) that were not converted in the olefin cracking reactor to the desired light olefins. This intermediate boiling fraction generally further comprises C₅-C₁₁ diolefin byproducts of the dehydrogenation and/or olefin cracking zones. An increase in the overall production of light olefins is therefore possible by selectively hydrogenating or saturating these diolefins, in a selective hydrogenation zone, to monoolefins and then cracking at least a portion of the monoolefins, generated in this manner, in the olefin cracking zone. All or a portion of the selective hydrogenation effluent may be passed to the olefin cracking reactor, in combination with the dehydrogenation effluent and/or a recycled portion of the heavy hydrocarbon byproduct as described above. A purge of a non-recycled portion of the intermediate boiling fraction is normally desired to prevent the excessive accumulation of intermediate-boiling (e.g., C₄-C₈) paraffins that are not otherwise easily removed from the process. These intermediate-boiling paraffins are normally present in relatively small quantities in the olefin cracking effluent, as a result of not being converted in the upstream reforming and/or dehydrogenation zone(s) or otherwise being generated as byproducts of the olefin cracking and/or selective hydrogenation zone(s). In alternative embodiments, all or a portion of the intermediate boiling fraction that is not sent to the selective hydrogenation zone is returned to the dehydrogenation zone together with the hydrocarbon feedstock entering this zone.

A representative, conventional selective hydrogenation catalyst for the conversion of diolefins to monoolefins in the selective hydrogenation zone comprises nickel and sulfur dispersed on an alumina support material having a high surface area, as described, for example, in U.S. Pat. No. 4,695,560. Selective hydrogenation is normally performed with the selective hydrogenation zone being maintained under relatively mild hydrogenation conditions, such that the hydrocarbons are present in the liquid phase and hydrogen can dissolve into the liquid. Suitable conditions in the selective hydrogenation zone include an absolute pressure from about 280 kPa (40 psia) to about 5500 kPa (800 psia), with a range from about 350 kPa (50 psia) to about 2100 kPa (300 psia) being preferred. Relatively moderate selective hydrogenation zone temperatures, for example, from about 25° C. (77° F.) to about 350° C. (662° F.), preferably from about 50° C. (122° F.) to about 200° C. (392° F.), are representative. The LHSV is typically greater than about 1 hr⁻¹, and preferably greater than about 5 hr⁻¹ (e.g., between about 5 and about 35 hr⁻¹). An important variable in selective hydrogenation is the ratio of hydrogen to diolefins, in this case present in the intermediate boiling fraction taken as a side draw from the fractionator (e.g., depropanizer), as discussed above. To avoid the undesired saturation of a significant proportion of the monoolefins, generally less than about 2 times the stoichiometric hydrogen requirement for diolefin saturation is used.

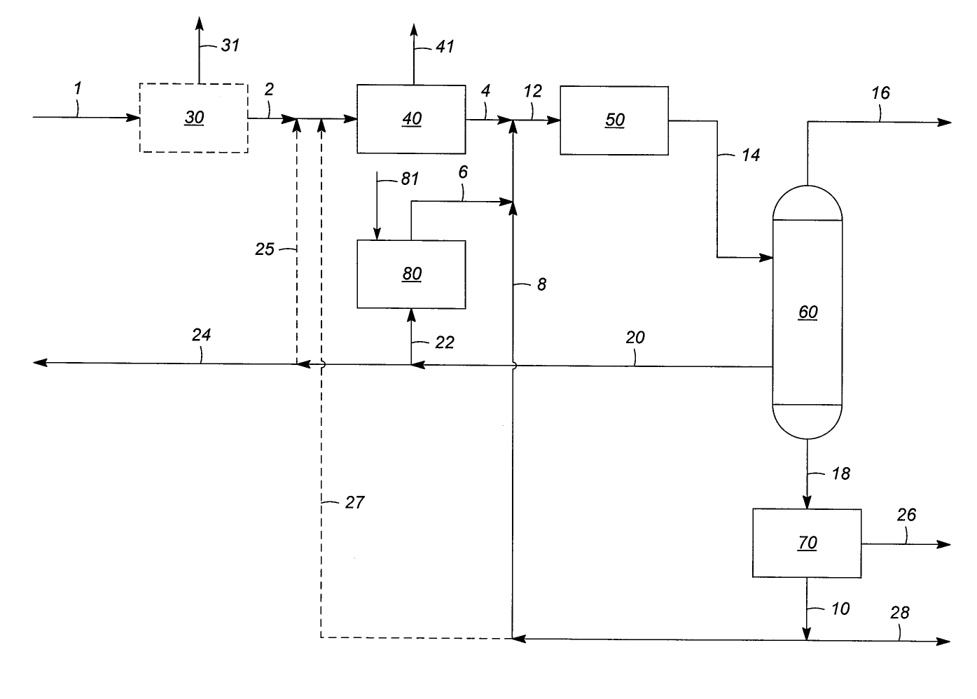

A representative process flowscheme illustrating a particular embodiment for carrying out the methods described above is depicted in FIG. 1. According to this embodiment, hydrocarbon feedstock 2 comprising paraffins in the C₅-C₁₁ carbon number range are passed to dehydrogenation zone 40 to provide dehydrogenation effluent 4 comprising olefins in this carbon number range. As discussed above, hydrocarbon feedstock 2 preferably comprises naphtha or, in some cases, a naphtha reforming effluent having undergone upstream reforming (e.g., CCR reforming) in reforming zone 30. Therefore, in the optional embodiment in which reforming zone 30 is used, reforming feed 1 preferably comprises naphtha.

Dehydrogenation effluent 4 is passed to olefin cracking zone 50 after optionally being combined with selective hydrogenation effluent 6 and/or recycled portion 8 of heavy hydrocarbon byproduct 10. At least a portion of the C₅-C₁₁ carbon number range olefins in dehydrogenation effluent 4, and also in combined olefin cracking zone feed 12, are then cracked in olefin cracking zone 50. Olefin cracking effluent 14 therefore comprises cracked, light olefins, namely propylene and ethylene, as well as C₆-C₉ aromatic hydrocarbons present in hydrocarbon feedstock 2, generated in dehydrogenation zone 40, and optionally generated in reforming zone 30. Olefin cracking effluent 14 is then passed to depropanizer 60 to provide low boiling fraction 16 containing substantially all of the propylene and ethylene and highly enriched in these hydrocarbons. High boiling fraction 18, for example as a bottoms stream of depropanizer 60, is enriched in aromatics, and intermediate boiling fraction 20 is taken as a side draw from depropanizer 60 and comprises unconverted olefins in the C₅-C₁₁ carbon number range, as well as byproduct diolefins in this carbon number range.

First recycled portion 22 of intermediate boiling fraction 20 is passed to selective hydrogenation zone 80, while first non-recycled portion 24 is purged to limit the accumulation of undesired byproducts such as paraffins that co-boil with intermediate boiling fraction 20. Optionally, second non-recycled portion 25 of intermediate boiling fraction 20 is returned to dehydrogenation zone 40 together with the hydrocarbon feedstock 2 entering this zone. In any event, at least a portion of the diolefins in intermediate boiling fraction 20 are therefore converted to monoolefins in selective hydrogenation zone 80, and these monoolefins are then passed in selective hydrogenation effluent 6 to olefin cracking zone 50 to enhance the overall yield of the light olefins propylene and ethylene. As discussed above, selective hydrogenation effluent 6 is combined with dehydrogenation effluent 4 and optionally recycled portion 8 of heavy hydrocarbon byproduct 10 upstream of olefin cracking zone 50. Selective hydrogenation zone 80 typically operates with a hydrogen addition stream 81 that provides an amount of hydrogen in excess of the stoichiometric amount for saturation of diolefins in recycled portion 22 of intermediate boiling fraction 20. Hydrogen addition stream 81 may be of varying purity and originate from various sources. For example, hydrogen addition stream 81 may comprise at least a portion of reforming zone net hydrogen product 31 and/or dehydrogenation zone net hydrogen product 41, optionally after purification of one or both of these products to increase hydrogen purity.

Aromatics in high boiling fraction (e.g., bottoms) of depropanizer 60 may be separated in aromatics recovery zone 70 to provide aromatic product 26 that is further enriched in aromatics and heavy hydrocarbon byproduct 10 that is depleted in aromatics. As discussed above, aromatics recovery zone 70 may utilize a physical solvent such as tetrahydrothiophene dioxide or rely on any other conventional means for separating aromatics from non-aromatic (aliphatic) hydrocarbons, which preferentially report to heavy hydrocarbon product 10. As shown in the embodiment of FIG. 1, recycled portion 8 of heavy hydrocarbon product 10 is combined with dehydrogenation effluent 4 prior to being passed to olefin cracking zone 50. Non-recycled portion 28 of heavy hydrocarbon product 10 is purged from the process to limit the accumulation of unwanted heavy hydrocarbon byproducts. As discussed above, a separate portion 27 of the heavy hydrocarbon byproduct that is not sent to the olefin cracking zone 50 is instead recycled to dehydrogenation zone 40 together with the hydrocarbon feedstock 2 entering this zone.

Overall, aspects of the invention are directed to processes for making propylene, ethylene, and aromatics comprising dehydrogenating naphtha or a naphtha reforming effluent in the presence of a catalyst comprising zirconia to provide a dehydrogenation effluent and cracking olefins in the dehydrogenation effluent. In view of the present disclosure, it will be seen that several advantages may be achieved and other advantageous results may be obtained. Those having skill in the art will recognize the applicability of the methods disclosed herein to any of a number of dehydrogenation/olefin cracking processes, and especially in the case of feeds comprising paraffins, naphthenes, and aromatics in the C₅-C₁₁ carbon number range. Those having skill in the art, with the knowledge gained from the present disclosure, will recognize that various changes could be made in the above processes without departing from the scope of the present disclosure. Mechanisms used to explain theoretical or observed phenomena or results, shall be interpreted as illustrative only and not limiting in any way the scope of the appended claims.

The following example is set forth as representative of the present invention. This example is not to be construed as limiting the scope of the invention as other equivalent embodiments will be apparent in view of the present disclosure and appended claims.

### EXAMPLE 1

Computerized yield estimating models were used to predict product yields obtained from the process flow schemes depicted in FIG. 1, both without (Case 1) and with (Case 2) upstream reforming of a model naphtha feedstock. The dehydrogenation zone was modeled based on pilot plant results obtained using a zirconia-based catalyst. The product yields were compared to a reference technology, namely catalytic naphtha cracking (Case 3), which does not generate aromatic hydrocarbons. The naphtha feed rate chosen as a basis for each simulation was 2,100 metric tons per year. Product hydrocarbon yields are summarized below in Table 1.

TABLE 1
Estimated Yields based in 2,100 MTA Naphtha Feed

| | Case 1, No | Case 2, Upstream | Case 3, |
|---|---|---|---|
| | Reforming | Reforming | Reference |
| | Mass % | Mass % | Mass % |
| Hydrogen | 3.32 | 3.82 | 1.56 |
| Methane | 2.46 | 2.77 | 8.51 |
| Ethane | 2.41 | 3.09 | 3.60 |
| Ethylene | 12.26 | 12.32 | 34.55 |
| Propane | 2.25 | 3.68 | |
| Propylene | 50.38 | 44.68 | 37.42 |
| C4s | 2.46 | 0.39 | 0.00 |
| Light Naphtha | 3.70 | 0.72 | 0.00 |
| Benzene | 4.86 | 9.50 | |
| Toluene | 6.18 | 9.18 | |
| Xylene | 5.43 | 6.11 | |
| Heavies Purge | 4.29 | 3.73 | |
| Reformate | | | 13.93 |
| Total | 100 | 100 | 99.57 |
| Prod/Feed Mass | 1.017 | 1.005 | 1.000 |

The yield estimation results show favorable yields of propylene, ethylene, and aromatics, both with and without optional, upstream reforming (Cases 1 and 2). Additionally, the inventive processes yielded light olefins with a significantly higher propylene:ethylene molar ratio, compared to the reference catalytic naphtha cracking process. Therefore, a relative increase in propylene demand/pricing would further improve the commercial attractiveness of the processes described herein over prior art processes. The processes described herein, according to various embodiments of the invention, are easily tailored to a wide variety of hydrocarbon feedstocks, including naphtha streams and naphtha reforming effluents having varying compositions. Value added products are obtained in the inventive processes from the conversion of both ring and non-ring hydrocarbons.

1. A process for the production of light olefins and aromatics, the process comprising:
(a) dehydrogenating a hydrocarbon feedstock comprising paraffins in a dehydrogenation zone to provide a dehydrogenation effluent comprising olefins;
(b) cracking at least a portion of the olefins in an olefin cracking zone to provide an olefin cracking effluent comprising ethylene, propylene, and aromatics.

2. The process of claim 1, wherein step (a) is carried out in the presence of a dehydrogenation catalyst comprising zirconia.

3. The process of claim 2, wherein the hydrocarbon feedstock comprises naphtha.

4. The process of claim 3, wherein the naphtha comprises hydrocarbons boiling in the range from about 100° C. (212° F.) to about 180° C. (356° F.).

5. The process of claim 4, wherein the naphtha comprises a total amount of C₅-C₁₁ carbon number range paraffins from about 40% to about 80% by weight.

6. The process of claim 2, wherein the hydrocarbon feedstock comprises a naphtha reforming effluent.

7. The process of claim 6, wherein the naphtha reforming effluent is obtained from reforming naphtha comprising a total amount of cyclopentane and alkylated cyclopentanes from about 10% to about 25% by weight.

8. The process of claim 2, wherein the olefin cracking effluent comprises propylene and ethylene in a propylene:ethylene molar ratio of at least about 2:1.

9. The process of claim 2, wherein the aromatics comprise benzene, toluene, and xylenes, which are present in the olefin cracking effluent in a total amount from about 10% to about 30% by weight.

10. The process of claim 2, further comprising:
(c) fractionating the olefin cracking effluent to provide fractions comprising a low boiling fraction enriched in the propylene and ethylene and a high boiling fraction enriched in the aromatics.

11. The process of claim 10, wherein the fractions further comprise an intermediate boiling fraction comprising olefins and diolefins in the C₅-C₁₁ carbon number range.

12. The process of claim 11, further comprising:
(d) selectively hydrogenating at least a portion of the diolefins in a selective hydrogenation zone; and
(e) cracking, in the olefin cracking zone, at least a portion of olefins obtained from selectively hydrogenating in step (d).

13. The process of claim 10, further comprising:
(d) separating the high boiling fraction into an aromatic product further enriched in the aromatics and a heavy hydrocarbon byproduct; and
(e) recycling at least a portion of the heavy hydrocarbon byproducts to the olefin cracking zone.

14. The process of claim 2, wherein step (a) is carried out under dehydrogenation conditions including an average dehydrogenation catalyst bed temperature from about 500° C. (932° F.) to about 700° C. (1292° F.), and an absolute pressure from about 50 kPa (7 psia) to about 2 MPa (290 psia).

15. The process of claim 2, wherein step (b) is carried out under olefin cracking conditions including an olefin cracking catalyst bed inlet temperature from about 400° C. (752° F.) to about 600° C. (1112° F.), and an absolute olefin partial pressure from about 110 kPa (1.5 psia) to about 200 kPa (29 psia).

16. An integrated dehydrogenation/olefin cracking process comprising:
(a) passing a hydrocarbon feedstock comprising paraffins in the C₅-C₁₁ carbon number range to a dehydrogenation zone to provide a dehydrogenation effluent comprising olefins in the C₅-C₁₁ carbon number range;
(b) passing the dehydrogenation effluent to an olefin cracking zone to crack at least a portion of the olefins and provide an olefin cracking effluent comprising ethylene, propylene, and aromatics;
(c) passing the olefin cracking effluent to a depropanizer to provide fractions comprising a low boiling fraction enriched in the propylene and ethylene, a high boiling fraction enriched in the aromatics, and an intermediate boiling fraction comprising olefins and diolefins in the C₅-C₁₁ carbon number range;
(d) passing at least a portion of the intermediate boiling fraction to a selective hydrogenation zone to provide a selective hydrogenation effluent comprising monoolefins obtained from the selective hydrogenation of at least a portion of the diolefins;
(e) combining the selective hydrogenation effluent with the dehydrogenation effluent prior to step (b).

17. The process of claim 16, further comprising:
(f) separating the aromatics in the high boiling fraction to provide an aromatic product further enriched in the aromatics and a heavy hydrocarbon byproduct; and
(g) combining at least a portion of the heavy hydrocarbon byproduct with the dehydrogenation effluent prior to step (b).

18. The process of claim 17, further comprising:
(h) purging non-recycled portions of the intermediate boiling fraction and the heavy hydrocarbon byproduct.

19. The process of claim 16, wherein the low boiling fraction is a light olefin product having a propylene:ethylene molar ratio of at least about 2:1.

20. A process for making propylene, ethylene, and aromatics comprising:
(a) dehydrogenating naphtha or a naphtha reforming effluent in the presence of a catalyst comprising zirconia to provide a dehydrogenation effluent and;
(b) cracking olefins in the dehydrogenation effluent.
